(19) United States
(12) Patent Application Publication
(10) Pub. No.: US 2016/0073887 A1
(43) Pub. Date: Mar. 17, 2016

(54) OPTOELECTRONIC DEVICE TO WRITE-IN AND READ-OUT ACTIVITY IN BRAIN CIRCUITS

(71) Applicant: Brown University, Providence, RI (US)

(72) Inventors: Joonhee LEE, Providence, RI (US); Arto V. NURMIKKO, Providence, RI (US); Yoon-Kyu SONG, Providence, RI (US)

(73) Assignee: BROWN UNIVERSITY, Providence, RI (US)

(21) Appl. No.: 14/783,741

(22) PCT Filed: Apr. 11, 2014
(86) PCT No.: PCT/US14/33765

§ 371 Date: Oct. 9, 2015

Related U.S. Application Data
(60) Provisional application No. 61/810,950, filed on Apr. 11, 2013.

(51) Int. Cl. A61B 5/00; A61B 5/0476; G02B 6/132; G02B 6/122; G02B 6/42; H01B 11/22; G02B 6/44; G02B 6/43; A61B 5/04; G02B 6/136

(52) U.S. Cl. CPC A61B 5/0084; A61B 5/04001; A61B 5/0476; G02B 6/132; G02B 6/136; G02B 6/4274; H01B 11/22; G02B 6/4416; G02B 6/43; G02B 6/122; A61B 2019/5206

(57) ABSTRACT

Systems, apparatus and methods for a neural implant are provided. In one embodiment, a neural implant that can both optically stimulate neurons and record electrical signals from neurons is provided, including a wide band gap semiconductor opto electronic microarray, such optoelectronic microarray including a plurality of needles, each providing both optical transparency and electrical conductivity; a flexible optical conduit from the optoelectronic microarray to an optical signal source; a flexible electrical conduit from the optoelectronic microarray to an electrical signal sensor; integration of the optical and electrical conduits to a single monolithic optical cable; a circuit assembly coupled to the electrical signal source and the optical signal source; and a processor for providing control of at least one of the electrical signal sensor and the optical signal source. Further embodiments are described herein.

### RELATED APPLICATIONS

This application claims the benefit of priority to U.S. Provisional Application No. 61/810,950 filed Apr. 11, 2013, the contents of which are hereby incorporated by reference in its entirety.

### GOVERNMENT SUPPORT

This work was supported by the Defense Advanced Research Projects Agency (DARPA) Reorganization and Plasticity to Accelerate Injury Recovery Program (REPAIR) (N66001-10-C-2010) and the National Science Foundation EFRI Grant (No. #0937848). The U.S. government has certain rights in this invention as provided for by the terms of the above grants.

### INCORPORATION BY REFERENCE

All patents, patent applications and publications cited herein are hereby incorporated by reference in their entirety in order to more fully describe the state of the art as known to those skilled therein as of the date of the invention described herein.

### BACKGROUND

This invention relates to implantable devices and methods for using such implants in a body.

Development of novel biomedical devices that can contribute and enable future bidirectional communication with the brain represents science and engineering at the very forefront of a major national thrust to understand brain computations in vivo. In addition to advancing fundamental brain science, a major rationale for developing such neurotechnological methods is the prospect of playing a major future role in assisting the large numbers of partially or severely disabled human patients with conditions such as paralysis, stroke, and refractory epilepsy, to name three cases where the use of cortical multielectrode array implants have been used in clinical trials and experiments respectively.

To advance neural prostheses and treatment of severe brain disorders, the next imperative is to “close the loop,” by enabling bi-directional communication with the brain with external electronics and patient-assistive medical device systems. Thus, in the case of a spinal cord injury, closing the loop could be in the form of delivering tactile sensation directly to the brain, an “artificial touch” percept to enable the paralyzed subject to operate a robotic hand at digit level control for, e.g., grasping a cup of coffee “by thought.” To make such artificial touch possible, a means of stimulation of the cortical circuits e.g. of the hand area in the sensory cortex is required.

### SUMMARY

Systems and methods for a neural implant are provided. In one embodiment, a neural implant providing both optical and electrical stimulation of neurons is provided, including a wide band gap semiconductor optoelectronic microarray, such optoelectronic microarray including a plurality of needles, each providing both optical transparency and electrical conductivity; a flexible optical conduit from the optoelectronic microarray to an optical signal source; a flexible electrical conduit from the optoelectronic microarray to an electrical signal source; integration of the optical and electrical conduits to a single monolithic optical cable; a circuit assembly coupled to the electrical signal source and the optical signal source; and a processor for providing control of at least one of the electrical signal source and the optical signal source. The neural implant may provide a plurality of optical channels and a plurality of electrical channels. In some embodiments, the neural implant may provide 100 channels and beyond. Further embodiments are described below.

### BRIEF DESCRIPTION OF THE FIGURES

FIG. 1 is a schematic diagram of a neural implant, in accordance with some embodiments.

FIG. 2 is a photograph of a neural implant, in accordance with some embodiments.

FIG. 3 is a micrograph of an optoelectronic microarray, in accordance with some embodiments.

FIG. 4 is a further schematic diagram of a neural implant, in accordance with some embodiments.

FIG. 5 is a photograph of a ZnO crystal, in accordance with some embodiments.

FIG. 6 is a schematic diagram of a ZnO crystal, in accordance with some embodiments.

FIG. 7 is a process flow diagram for dicing and preparation, in accordance with some embodiments.

FIG. 8 is a process flow diagram for dicing and etching, in accordance with some embodiments.

FIG. 9 is a process flow diagram for optoelectronic microarray tip metallization, in accordance with some embodiments.

FIG. 10 is a photograph of stress testing of a silicon-based microelectrode array, in accordance with some embodiments.

FIG. 11 is a photograph of stress testing of a ZnO-based microelectrode array, in accordance with some embodiments.

FIG. 12 is a photograph of a ZnO optoelectronic microarray with wirebundle, in accordance with some embodiments.

FIG. 13 is a photograph of a ZnO optoelectronic microarray with processor card, in accordance with some embodiments.

FIG. 14 is a micrograph of a ZnO optoelectronic microarray shown in a tilted view, in accordance with some embodiments.

FIG. 15 is a microscope image of a ZnO optoelectronic microarray, in accordance with some embodiments.

FIG. 16 is a recording of neural activity, in accordance with some embodiments.

FIG. 17 is a further recording of neural activity, in accordance with some embodiments.

FIG. 18 is a recording of an individual neural spike event, in accordance with some embodiments.

FIG. 19 is a further recording of an individual neural spike event, in accordance with some embodiments.

FIG. 20 is a further schematic diagram of an optoelectronic microarray, in accordance with some embodiments.

FIG. 21 is a further schematic diagram of a planar ribbon cable, in accordance with some embodiments.

FIG. 22 is a micrograph of a wet-edged optoelectronic microarray tip, in accordance with some embodiments.

FIG. 23 is a simulated tip emission pattern, in accordance with some embodiments.

FIG. 24 is a further simulated tip emission pattern, in accordance with some embodiments.

FIG. 25 is a top view of a 16-channel polyamide test cable, in accordance with some embodiments.

FIG. 26 is an isometric view of an optical waveguide, in accordance with some embodiments.

FIG. 27 is a cross-sectional micrograph of an optical waveguide, in accordance with some embodiments.

FIG. 28 is a schematic top view of an optoelectronic microarray electrical connection scheme, in accordance with some embodiments.

FIG. 29 is a schematic cross-sectional view of an optoelectronic microarray electrical connection scheme, in accordance with some embodiments.

FIG. 30 is a schematic view of an alternative embodiment of an optoelectronic microarray that comprises integrated light sources, in accordance with some embodiments.

### DETAILED DESCRIPTION

Neural prostheses can be used to enable patients to interface with the outside world directly, through the prosthesis. In case of a quadriplegic patient, for example, a multi-electrode array extracts information about the neural code which, after decoding by statistical data-driven algorithms, has been able to translate a patient's thoughts into usable electronic command of a robotic arm/hand. In this case, access to neural circuit dynamics at single neuron level using chronic intracortical implants can record action potentials from typically 100 sites at a 1-2 mm depth in a given brain area, such as the hand and arm areas of the primary motor cortex. Similar human clinical trials and experiments are being pursued by several groups across the U.S. while recording neural population dynamics for translating thought into action.

While the present disclosure describes the implantation of devices in rodents, various embodiments are understood to include implantation of devices in non-human primates as well as humans.

To advance neural prostheses and treatment of severe brain disorders, the next imperative is to “close the loop,” by enabling bi-directional communication with the brain with external electronics and patient-assistive medical device systems. Thus, in the case of a spinal cord injury, closing the loop could be in the form of delivering tactile sensation directly to the brain, an “artificial touch” percept to enable the paralyzed subject to operate a robotic hand at digit level control for, e.g., grasping a cup of coffee “by thought.” To make such artificial touch possible, a means of stimulation of the cortical circuits e.g. of the hand area in the sensory cortex is required. In case of a severely epileptic patient, research suggests that listening to brain circuits can provide a ‘warning indicator’ prior to the onset of seizures, whereby intervening by stimulation (inhibition) of a target brain area may suppress an epileptic episode. Advancing reliable chronic human neuroprostheses as complex biomedical engineering systems does face challenges at several different levels, of which the brain implant device is one piece of a larger puzzle.

Electrical microstimulation has a venerable history in neuroscience, including clinical use in stimulating deeper brain areas (DBS) such as the subthlamic nucleus (STN) to control tremors in patients with Parkinsonian tremor. However, intracortical electrical stimulation in a closed-loop brain-machine interface system has significant drawbacks because (i) it is not usually spatially specific at digit level, and (ii) the electrical noise from the stimulation interferes with the recording to make the latter very challenging in practice (stimulation currents several orders of magnitude larger than neural recording currents).

Within the past few years, a means of using visible light to stimulate well-defined brain targets called optogenetics has had a major impact on the field of neuroscience. Optogenetics involves the use of microbiological transduction means to convert a small subset of e.g. targeted cortical neurons to become light-sensitive, within a volume of 1 mm³. By engineering the ion channel opsin protein, both excitation and inhibition of neural cell activity has been achieved. The genetic DNA materials behind the opsins, such as channelrhodopsin and halorhodopsin, have their biological origin in smaller organisms whose energy uptake is provided by sunlight, mainly in the blue and in the green. Starting from mice as the first animal models around 2005, the technique has since been richly extended to other rodents and very recently cross-species transitioned to non-human primates.

A large number of photomodulation experiments in rodent animal models, a veritable explosion in neuroscience, have shown the ability of optogenetics to induce and study behavioral effects with exceptional clarity; the journal Nature named optogenetics as the method of the year in 2011. Specifically, the ability to simultaneously record signal from light triggered neurons and circuits in a well-defined targeted brain volume would give the brain science research community an entire set of new methods to study the effects of induced perturbations on neural circuits and networks We now have the tantalizing prospect of translating this basic research to clinical applications.

One experimental device arrangement in optogenetics research on in-vivo animal models deploys an optical fiber as a means of delivering photoexcitation to optogenetically transduced volumes. An optical fiber can be combined with a microelectrode (typically a microwire, or a micromachined silicon shank) for making an “optical electrode”, by simply physically attaching the two side by side. The number of sites (channels) can be increased in both optical stimulation and electrical recording. However, due to materials and fabrication problems none of these constructs are likely to be scalable to reach anywhere near the ultimate goal of a 100-channel (and beyond) joint optical stimulation and electrical recording. Further, there is a fundamental material incompatibility at issue with both silicon and metal microwires being optically opaque. To obtain bidirectional performance where multiple neurons within a given functional circuit can be accessed by precisely-patterned spatio-temporally specific inputs/outputs in chronic use may require an integrated, monolithic device. This is dual-functional in that each element of such an array provides both optical and electrical access to the very same spatial target, and can be used for both optical stimulation and electrical recording across the entire array. In this proposal, we call such the device a optoelectronic microarray (OEM), which may meet the robust demands of chronic implant performance, and may also be compatible with a mobile subject.

We have developed practically useful optoelectronic microarray (OEMs) for both excitatory and inhibitory neural circuit modulation and augmenting sensory and motor signaling based on spatially and temporally patterned optical stimulation of optogenetically transduced targeted cortical areas of the brain. The wide band gap semiconductor OEMs can be chronically viable, and are suitable for a durable, high-performance brain implant.

To realize a monolithic, integrated cortical multichannel implant for using light to input information to cortical circuits while recording from the corresponding neural circuit, a specific class of so-called wide-band gap crystalline semiconductors may be used as the biocompatible materials platform. Wide-band gap inorganic semiconductors based on materials such as zinc oxide, gallium nitride, and silicon carbide have the unusual combinatorial attributes of optical transparency and high electrical conductivity.

The field of optogenetics is today breaking from its initial explosive growth in basic neuroscience to a number of potentially significant biomedical engineering directions. These include brain-machine interfaces with closed-loop control and the ability to target neurons and neural circuits for controlling and modulating an errant brain in cases of refractory neurological illnesses. As basic and applied work is proceeding with in-vivo animal models, with such human health questions looming in background, the research community is actively searching for devices that will expand the opportunities for “writing-in” and “reading-out” neural circuit information from the living brain. The disclosed embodiments offer a neural implant concept and device construct, which satisfies most of the idealized goals for an intracortical implant to operate chronically in the dual-function mode while engaging neural circuits of specific interest and function. The disclosed optoelectronic microarray (OEM) platform is scalable in a number of ways, where in the form of multiple arrays, reconfiguration as ECoG arrays, and so on. Finally, the disclosed devices can integrate directly into leading edge neuroprosthesis and neural diagnostic systems, hopefully thus impacting the broader field of upcoming neurotechnologies.

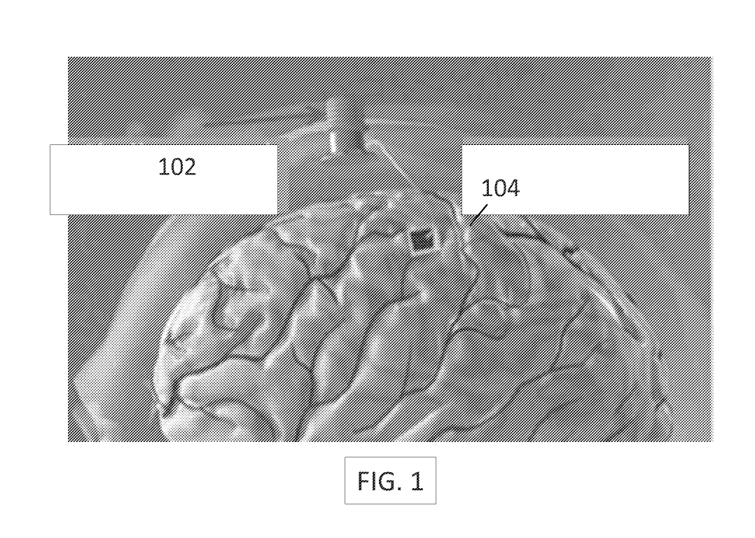

FIG. 1 is a schematic diagram of a neural implant, in accordance with some embodiments. Skull-mounted pedestal 102 provides a physical interface between an intracranial region and an extracranial region. Microelectrode array 104 is located in the intracranial region, i.e., within the body, within the brain, and within the cortex. Skull-mounted pedestal may be a titanium percutaneous pedestal, in some embodiments.

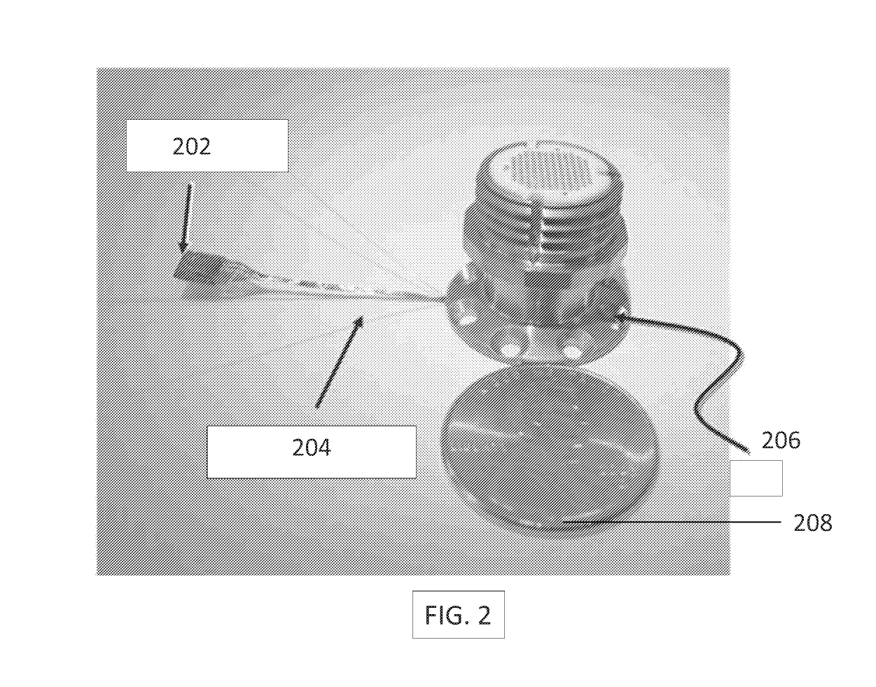

FIG. 2 is a photograph of a neural implant, in accordance with some embodiments. Microelectrode array 202 is coupled to cable 204, which is coupled to brain interface device 206. Penny 208 is presented to provide a size reference. Microelectrode array 202 may be a Utah-model microelectrode array (MEA). Brain interface device 206 may include a skull-mounted pedestal as described above at 102, and may also include additional circuitry for processing neural signals, as in, for example, Patent Cooperation Treaty (PCT) App. No. PCT/US2012/29664, “Implantable Wireless Neural Device,” which is hereby incorporated by reference in its entirety. Brain interface device 206 may be a titanium percutaneous pedestal, in some embodiments.

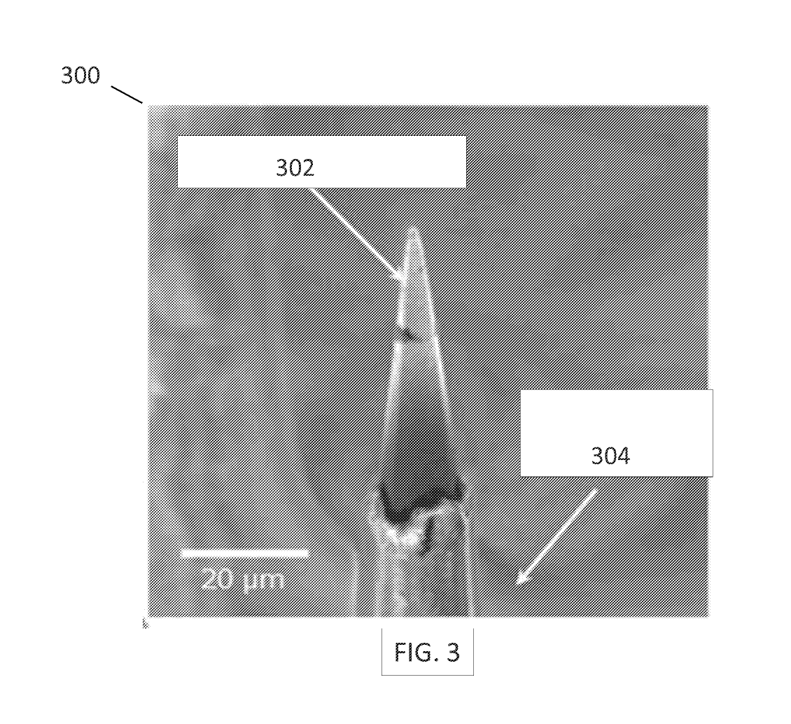

FIG. 3 is a micrograph of an optoelectronic microarray, in accordance with some embodiments. Optoelectronic microarray tip 302 may be coated with a special coating, which in some embodiments may be a optically transparent thin film additional coating such as indium-tin oxide (ITO) or indium zinc oxide (IZO). Optoelectronic microarray body 304 may be coated by additional dielectric to define the precise area of the portion of the conductive body which contacts electrically and optically to brain tissue. in some embodiments the coating may be made of insulating thin film such as parylene and/or alumina.

FIGS. 1-3, as described above, show images of and depiction of use of the “Utah” micro-electrode array, in accordance with some embodiments. The depicted MEA can be used as an intracortical sensor device from rodents to primates (Blackrock Inc. Salt Lake). Regarding multielectrode arrays (MEA), intracortical arrays have the particularly useful feature of being able to “listen” to neural circuits dynamics at single cell resolution. Different geometrical configurations exist such as those defined by microwire arrays and silicon-based probes of the “Utah” and “Michigan” types, among others. (See the image of the Utah array in FIG. 2). In context of human neuroprostheses as well as epilepsy monitoring, subdural or epidural electrocortical grids (ECoG) offer an alternative device, though at much lower spatial and temporal resolution (detecting field potentials as opposed to “spikes” of action potentials). We focus in this disclosure on the “Utah” form factor MEAs, largely since these sensors have been used at the inventors' home institution for nearly 20 years, as chronic implants across animal models to recent human clinical trials; however other array geometries are also contemplated. These MEAs have the advantage that the tapered shanks of the individual OEM elements act as natural light focusing guides for photoexcitation to be released at the very tip of the elements into nearby cortical domain. However, the proposed OEMs are not fundamentally limited to a specific geometry.

Wide band gap semiconductors such as group II-VI compound semiconductors ZnSe and ZnO, group III-V compounds such as GaN, and group IV compound SiC have unusual properties. They are transparent across the visible to the blue and near-ultraviolet while benefiting from robust electrical conductivity. Wide band gap semiconductors show high optical transmittance from ultraviolet to infrared with controllable electrical properties by doping and annealing. Among them, the biologically compatible II-VI compound ZnO was has been used by us as an optoelectronic proof-of-concept brain implant device compatible with fabrication via specific microelectronic process techniques which we have developed.

In one or more embodiments, the semiconductor substrate is doped to provide electrical conductivity. The semiconductor substrate remains optically and electromagnetically transparent after doping. While the n-type, electron-rich doping of wide band gap semiconductors is relatively straightforward, p-type doping is not. These compound semiconductors are seeing broad use as the backbone of blue and green light emitters, as high power RF amplifiers, and as piezoelectric transducers across today's electronics technologies. On the other hand, their other physical properties make these materials complex and challenging to fabricate without extensive experience.

Initial steps have been made into assessing the compatibility and microfabrication of ZnO, GaN, and SiC, respectively, with ZnO being selected as the proof-of-concept material candidate. An added factor in the material choice proposed here was the commercial availability of bulk ZnO substrates (at least 1-2 mm thick) as opposed to the much more common form of epitaxial thin films (up to maximum of about 10 μm thick). We next describe the method of Zno-based OEM fabrication.

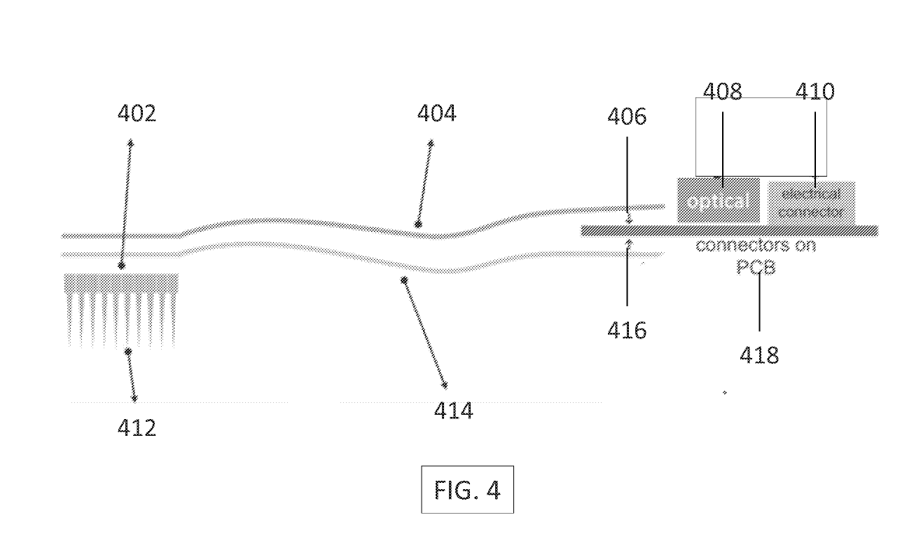

FIG. 4 is a further schematic diagram of a neural implant, in accordance with some embodiments. FIG. 4 shows several components of a chronic OEM. This consists of two major device components aims: an optoelectrical; array, which is the ZnO OEM itself, and a flexible dual-function connector cable which contains both multichannel electrical and optical “wirebundles,” including methods to reliably join the cable to the OEM as well as at its distal end. The purpose of the integrated flexible cable is to both guide light in and extract electrical neural signals out from the intracortical array, threaded through the subject's skull, onto a skull mounted pedestal for connection to external electronics, as in and multiple works on rodents and non-human primates, or to a subcutaneous wireless body implant for example. Many different pedestals or their wireless equivalents could be used, and a variety of light sources (blue green LEDs vs. compact solid state lasers) could also be used. In one embodiment, multi-element blue LEDs are used in conjunction with imaging optical fibers.

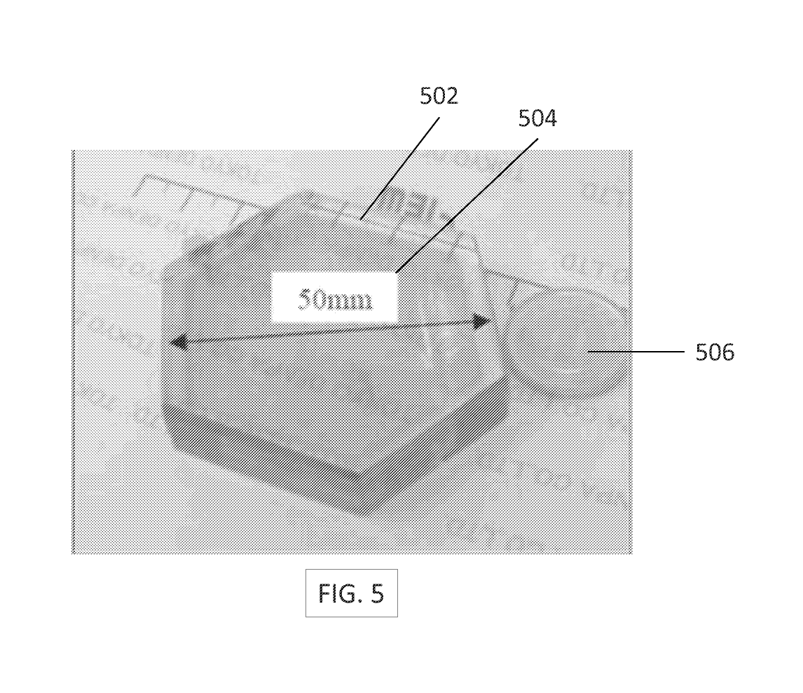

FIG. 5 is a photograph of a ZnO crystal, in accordance with some embodiments. A starting ZnO single crystal block material 502 can be used. The crystal block material 502 can be acquired from a commercial vendor. The starting ZnO single crystal block material 502 is grown by a hydrothermal process to a size and diameter of 50 mm as shown by label 504 and as referenced by Japanese 1-yen coin 506. The ZnO bulk single crystal material 502 can also be validated for its optical transparency and electrical conductivity to meet various specifications, in some embodiments. The specifications may include optical transparency, electrical conductivity, electrical impedance, and structural integrity in the desired physical shape and configuration. In some embodiments, multiple ZnO crystals can be used as the starting material. In some embodiments, the dimensions of the finished optoelectronic microarray can be in the range of 20-200 μm. In some embodiments, the size and shape of the finished optoelectronic microarray can be dictated by the size and geometry of the target organism's brain.

FIG. 5 is a diagram of the starting single crystal ZnO block material and the relevant crystalline orientation of its facets, in accordance with some embodiments. FIG. 5 shows an image of the starting ZnO single crystal block, grown by a hydrothermal process. Several different routes were explored for a compatible device processing route, whereby a 100 element square optoelectronic microarray could be carved from the solid block in the form of electrically and optically isolated elements. A “Utah MEA” geometry was chosen, as this has the double advantage of not only providing electrical recording from the tips of the “needles” but that the tapered geometry acts naturally as a low-loss optical waveguide for the transparent wide gap semiconductors due to their dielectric properties. Both computer simulations and experiments have shown that after entering an approximately 200×200 μm area at the base of an individual OEM element, the tapering enables light to exit into adjacent neural tissue from an aperture of about 10 micrometers—a good value from viewpoint of spatially specific optical targeting but demanding the development of a specific etching recipe which exploits the anisotropic (hexagonal wurtzite) crystal structure of ZnO.

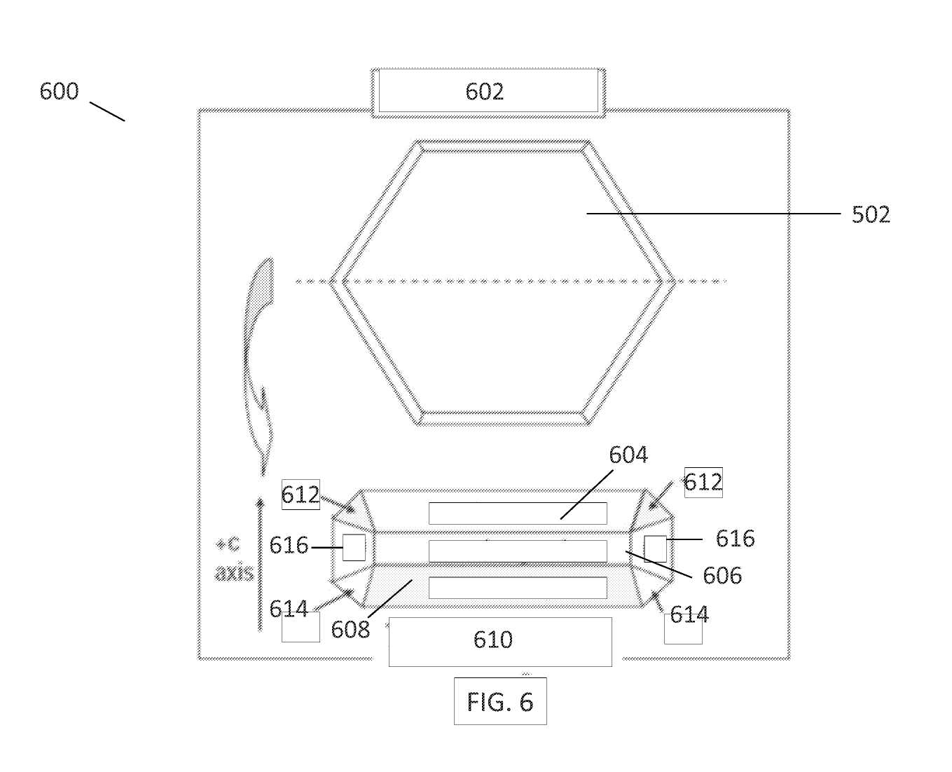

FIG. 6 is a schematic diagram of a ZnO crystal, in accordance with some embodiments, including the relevant crystalline orientation of its facets 600. Several different routes were explored for a compatible device processing route, whereby a 100 element square optoelectronic microarray could be carved from the solid block 502 in the form of electrically and optically isolated elements 602. FIG. 6 illustrates a cross section 610 of the “Utah MEA” geometry, including +c sector 604, seed crystal 606, −c sector 608, +p 612, m 616, and −p 614. The “Utah MEA” geometry can provide electrical recording from the tips of the “needles.” The tapered geometry can act naturally as a low-loss optical waveguide. In some embodiments, after entering an approximately 200×200 μm area at the base of an individual OEM element 602, the tapering enables light to exit into adjacent neural tissue from an aperture of about 10 micrometers. In some embodiments, this aperture can be located at a second, narrower end of the OEM element 602 that is distal to the base at which the light enters the element This aperture can provide spatially specific optical targeting. This aperture can be provided or enhanced by the use of an etching recipe that exploits the anisotropic (hexagonal wurtzite) crystal structure of ZnO 502.

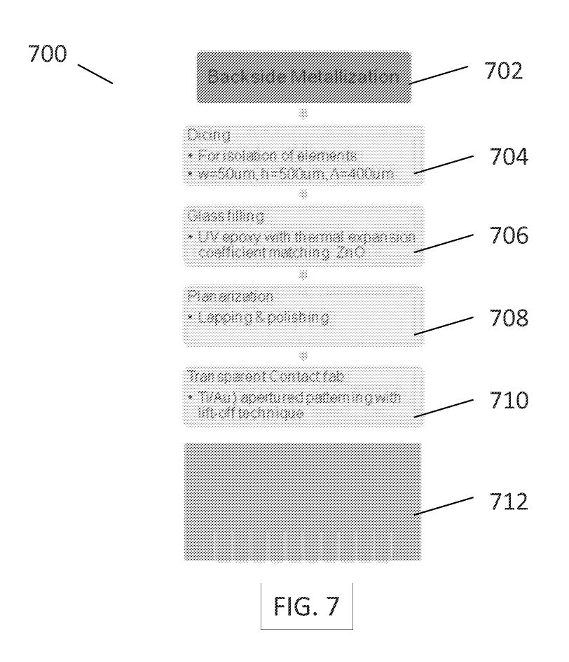

FIG. 7 is a process flow diagram for dicing and preparation, in accordance with some embodiments. Process flow diagram 700 shows the initial dicing and preparation of the “backside” of the OEM for electrical and optical isolation. Backside metallization 702, dicing 704, backside gap filling with isolating, adhesive material 706, planarization 708, and transparent contact fab 710 are shown. Schematic 712 is a representative schematic of the state of an optoelectronic microarray after undergoing 702-710. At 702, backside metallization can be performed. At 704, dicing is performed for isolation of elements, using parameters that may include w=50 μm, h=500 μm, A=400 μm. At 706, the gap is filled with an isolating adhesive, such as glass, which may be using an ultraviolet (UV) epoxy. Other adhesive materials can be used, such as UV epoxy, and other polymer adhesives with a thermal expansion coefficient matching ZnO (or other wide band gap semiconductor that is used). At 708, planarization, including lapping and polishing, can be performed. At 710, transparent contact fabrication can be performed, including fabrication of a Ti/Au-apertured patterning in some embodiments, and using a lift-off technique in some embodiments.

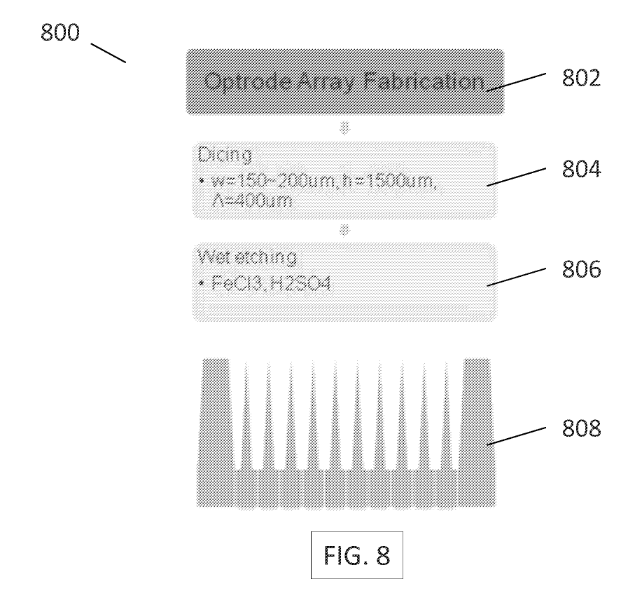

FIG. 8 is a process flow diagram for dicing and etching, in accordance with some embodiments. Process flow diagram 800 shows dicing and chemically anisotropic but controlled etching of the actual “needles” of the OEM by specific wet chemistry. Flow diagram 800 shows optoelectronic microarray fabrication 802, dicing 804, and wet etching 806. In some embodiments, wet etching 806 can be performed using FeCl3 and H2SO4. Schematic diagram 808 is a representative schematic of the state of an optoelectronic microarray after undergoing 802-806.

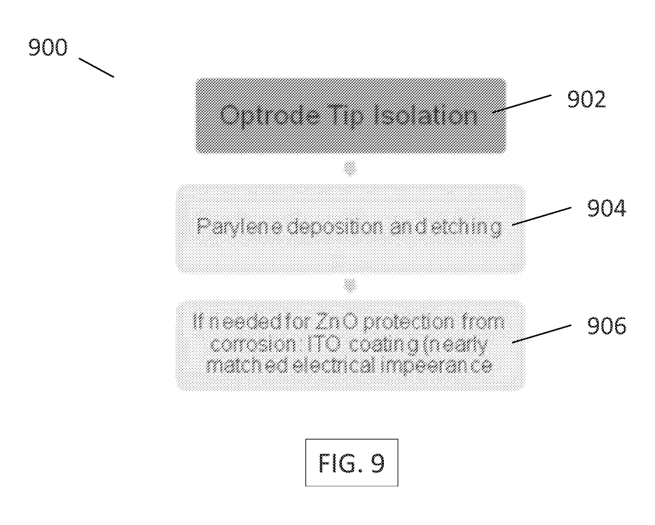

FIG. 9 is a process flow diagram for electrode tip metallization, in accordance with some embodiments. Process flow diagram 900 shows electrical contact metallization. Flow diagram 900 starts with electrode tip isolation 902 parylene deposition and etching 904, and finishing with 906 if needed for ZnO protection from corrosion. In some embodiments, the coating is an ITO coating, which provides nearly-matched electrical impedance.

We have tested the electrical recording capabilities of OEMs on bench top and in an animal (mouse), since demonstrating good extracellular response comparable that to the Pt-coated Si-electrodes was judged to be critical. On bench top electrical currents mimicking neural cell activity were injected into saline (mimicking the cerebrospinal fluid) and impedance measured. Our results have been electrode impedances in the range of several hundred Kohm to somewhat above 1 Mohm, which is the range desired for sensitive electrical measurements.

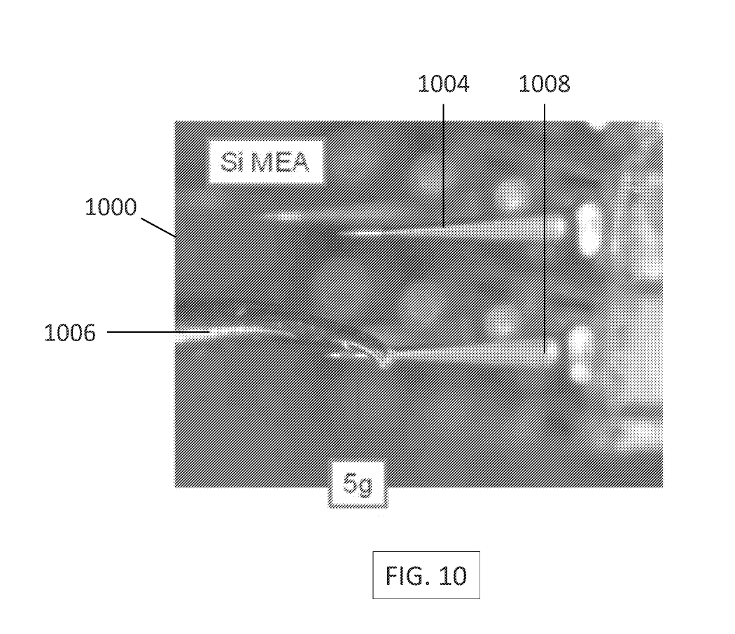

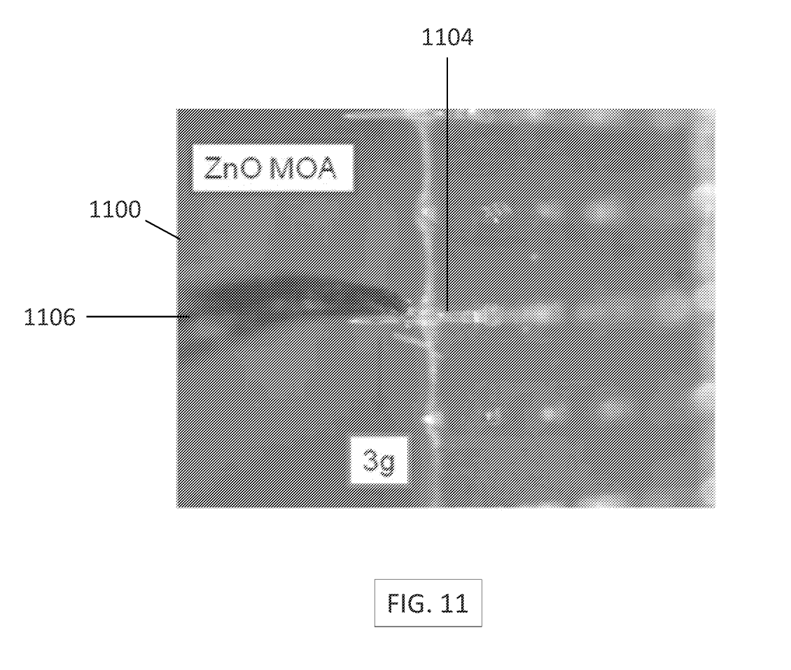

FIGS. 10-11 shows bending forces on a ZnO crystal, in accordance with some embodiments, while comparing ZnO with a nontransparent silicon-crystal “Utah” array.

FIG. 10 is a photograph of stress testing of a silicon-based microelectrode array, in accordance with some embodiments. Si multi-electrode array (MEA) 1000 includes electrodes 1004 and 1008. Electrode 1008 is touched by load test wire 1006. In some embodiments, load test wire 1006 may be a curved small-diameter wire, and load test wire 1006's positioning and load can be adjusted with a wirebond testing machine. In some embodiments, a force of 5 g can be put on electrode 1008 using load test wire 1006.

FIG. 11 is a photograph of stress testing of a ZnO-based microelectrode array, in accordance with some embodiments. ZnO optoelectronic microarray (OEM) 1000 includes electrode 1104. Electrode 1104 is touched by load test wire 1106. In some embodiments, load test wire 1106 may be a curved small-diameter wire, and load test wire 1106's positioning and load can be adjusted with a wirebond testing machine. In some embodiments, a force of 3 g can be put on electrode 1104 using load test wire 1106.

One concern with all single crystal bulk-based devices is their vulnerability to stress which can lead to physical breakage of the electrodes by cleavage along specific atomic planes. While the brain tissue itself is soft, concerns about breakage are real during the handling of the arrays, e.g. during implant surgery. We performed a basic “bend test” to compare the Si- and ZnO-based arrays. While the crystal structures (hence directions of preferential cleavage differ between cubic Si and hexagonal ZnO, FIGS. 10 and 11 show that the bending forces to reach breakage are comparable with ZnO of slightly the more fragile of the two.

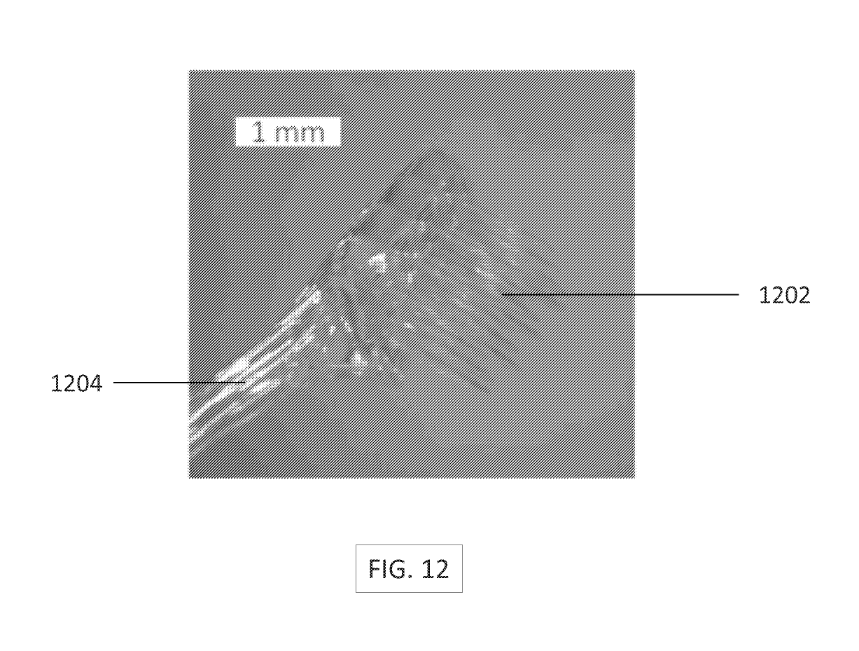

FIG. 12 is a photograph of a ZnO optoelectronic microarray with wirebundle, in accordance with some embodiments. Optoelectronic microarray 1202 is shown coupled to potted Au wirebundle 1204. In some embodiments, optoelectronic microarray 1202 is an electrically-wired 4×4 ZnO OEM prepared for an acute rat experiment.

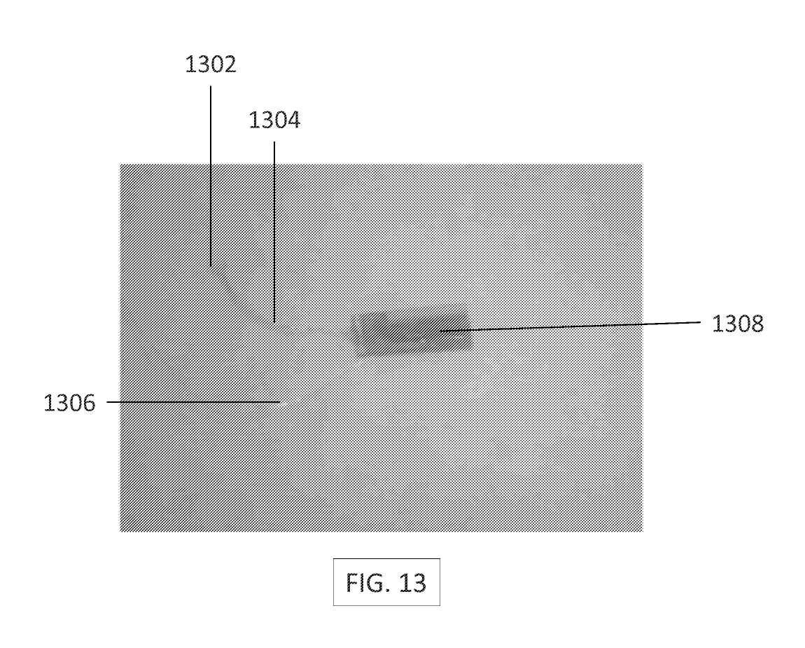

FIG. 13 is a photograph of a ZnO optoelectronic microarray with processor card, in accordance with some embodiments. Optoelectronic microarray 1302 is shown coupled to potted Au wirebundle 1304. Wirebundle 1304 is further coupled to neural signal amplifier/processor card 1308. Neural signal amplifier/processor card 1308 is also coupled with reference ground wire 1306. In some embodiments, optoelectronic microarray 1202 is an electrically-wired 4×4 ZnO OEM prepared for an acute rat experiment.

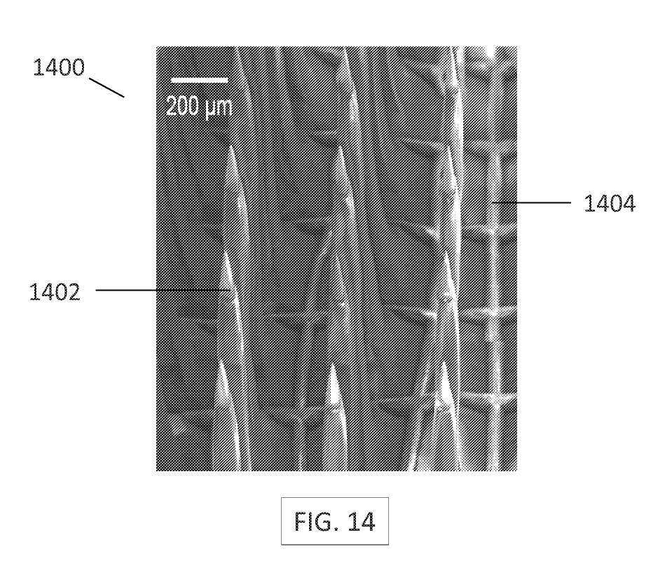

FIG. 14 is a micrograph of a ZnO optoelectronic microarray shown in a tilted view, in accordance with some embodiments. Micrograph 1404 is a tilted view of a ZnO OEM obtained using a scanning electron microscope. In some embodiments, optoelectronic microarray 1402 is coupled to substrate 1404.

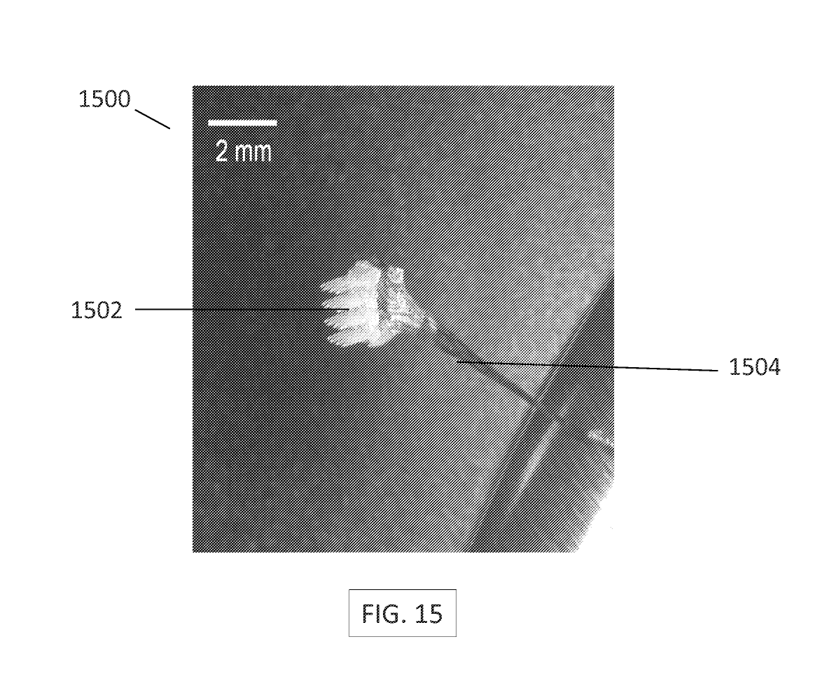

FIG. 15 is a microscope image of a ZnO optoelectronic microarray, in accordance with some embodiments. Microscope image 1500 shows ZnO optoelectronic microarray 1502 coupled to electrical cable 1504. In some embodiments, ZnO optoelectropic microarray 1502 is a fully-processed 4×4 OEM for in vivo chronic mouse implant use. In some embodiments, electrical cable 1504 is a ribbon electrical multi-channel cable or wirebundle. Further embodiments are also disclosed herein where an optical cable is co-located with the ribbon electrical multi-channel cable.

An in-vivo animal experiment was performed as follows: a transgenic mouse was chosen from the line Thy1-Chr2-YFP where widespread expression of the cortex by channelrhodopsin ChR2 makes this a useful first animal test of any new optical-electrical dual function device. The acute experiment was performed with the animal under anesthesia. Following a craniotomy, a 4×4 element ZnO OEM was inserted to the cortex. In this device, the electrical wiring of to the array was made by simple wedge bonding of the 25 micron wires—in turn cabled to an external connector to electronic instrumentation. FIGS. 12-13 shows a photograph of the optoelectronic microarray (1.5 mm long electrodes, 400 μm pitch) with its insulated gold wirebundle, as well as the view of the connection to a nearby printed-circuit board.

A device was created which enables simultaneous optical stimulation and electrical recording at single neuron resolution at multiple sites (up to 100-channels) across a neural microcircuit of interest. In other embodiments, the device can include any number of needles or other geometrical shapes conducive to simultaneous neural signal recording and stimulation, for example up to 16, up to 25, up to 49, up to 64, up to 81, up to 100 or up to 1000. For the device geometry we chose a planar intracortical 2D array similar to that used successfully with opaque Si-based multielectrode arrays from rodents to recent breakthrough human clinical trials.

Briefly, we began with a 2 mm-thick n-type, [0001]-oriented ZnO semiconductor single crystal (resistivity=0.15 Ω·cm). A 2D square array of Ti/Au wire bonding pads with 400 um pitch was patterned by photolithography and lift-off technique. Then, a 600 μm-deep, 50 μm-wide trench was formed between the pads by dicing ZnO substrate and filled with polymer adhesive. Opposite surface of the substrate was also diced down to form an electrically separated 1.5 mm-tall, 250×250 μm2 ZnO square pillar array.

To achieve a OEM with sharply tapered tip, specific and tightly-controlled multistep wet chemical etch processes were developed, among which Fe(III) chloride solution provided the anisotropic etch which resulted in truncated pyramid tip shape of the ZnO pillars. Then, diluted sulfuric acid with deionized water at 45° C. was used to control the tapered shape and surface roughness on micrometer scale. A thin layer of parylene-C film was next deposited on ZnO as an electrical insulation layer of individual electrodes at ambient temperature by evaporating parylene monomer. Parylene-C can be chosen to provide biocompatibility, pinhole-hole free conformal coating and dielectric properties.

Exposing of electrically and optically active tip region is a significant process step because its area affects the impedance value (Z) of the optoelectronic microarray. Uniform tip exposure was achieved by applying viscous poly(dimethylsiloxane) (PDMS) in a masking method. During the process, PDMS and Parylene-C layer were removed by a fluorine-based inductively-coupled plasma (ICP) etch process to define the exposed height and area of each element of the OEM on micrometer-scale accuracy and uniformity. Subsequently, transparent and conductive indium tin oxide (ITO) layer was sputtered on the ZnO surface. Finally, a flexible electrical interconnect was formed with insulated Au bonding wires embedded in a custom designed PDMS ribbon cable. Bonding wires in the each row of the OEM were vertically aligned to maximize open area for optical access.

For assessing the OEM's performance in electrophysiological recording, impedance spectroscopy data were obtained by probing each 1.5 mm long electrodes, penetrating 1 wt % agar in artificial cerebrospinal fluid solution (ACSF). The average impedance value for a 4×4 element rodent device at 1 kHz across the 16 of the optoelectronic microarray was 386±58 kΩ, indicating the electronic uniformity of the OEM. For our first in vivo optical experiments, we chose a transgenic mouse model. After implant, recordings were made from the posterior parietal cortex of anesthetized transgenic mice expressing Channelrhodopsin-2 (ChR2). Optical excitation of 1s continuous laser pulse (473 nm) was delivered through optical fiber proximate to the OEM to illuminate its full surface area (1.2×1.2 mm2) while simultaneously monitoring the brain activity.

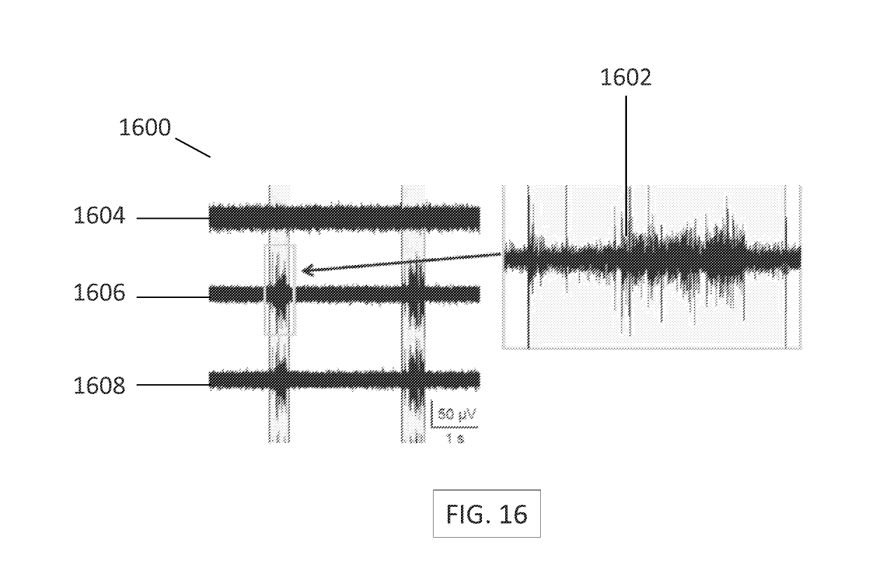

FIGS. 16-19 show light-induced (stimulated) neural activity recorded across several channels of the array, each “listening” to a single nearby neuron. A comparison with waveforms of single action potentials in spontaneous activity provided the single unit reference. The recorded signal was filtered from 300 to 1000 Hz so that any slow-changing photoelectrical artifacts could be removed.

FIG. 16 is a recording of neural activity, in accordance with some embodiments. Waveform diagram 1600 shows electrical activity measured from three neurons using a ZnO OEM, where activity is shown on the y-axis and time is shown on the x-axis. In some embodiments, the electrical activity is induced using 450 msec laser pulses in an anesthetized acute transgenic mouse. Channel 1604 shows no evoked activity and shows non-stimulated neural activity or background activity. In some embodiments, this may be due to a wedge-bonded Au wire being disconnected from a neuron, which may occur during insertion in some embodiments. Channel 1606 shows evoked activity. Channel 1608 also shows evoked activity on a separate channel. Inset 1602 shows a detailed view of evoked activity on channel 1606.

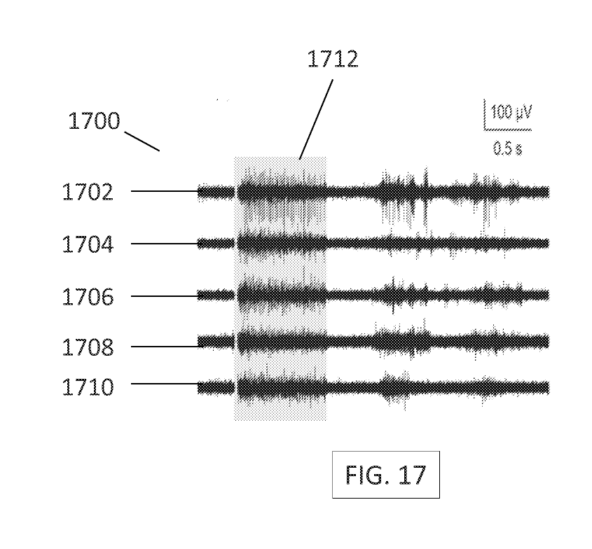

FIG. 17 is a further recording of neural activity, in accordance with some embodiments. Waveform diagram 1700 shows electrical activity measured from five neurons using a ZnO OEM, where activity is shown on the y-axis and time is shown on the x-axis. In some embodiments, the electrical activity is induced using laser pulses in a ChR2 transgenic mouse. Channels 1702, 1704, 1706, 1708, and 1710 each show evoked activity in the mouse evoked using a laser pulse. Box 1712 marks the extent in time of a 1-second continuous laser pulse used to evoke the neural activity shown.

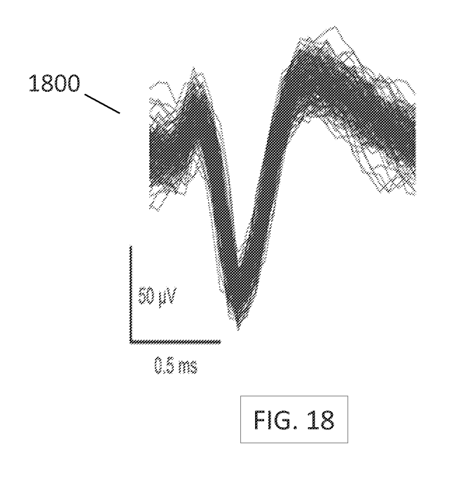

FIG. 18 is a recording of an individual neural spike event, in accordance with some embodiments. Neural spike recording 1800 is a spontaneous spike event that is not evoked using optical stimulation.

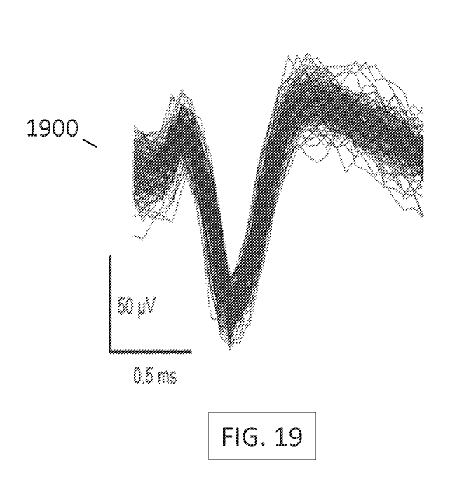

FIG. 19 is a further recording of an individual neural spike event, in accordance with some embodiments. Neural spike recording 1900 is a spontaneous spike event that is evoked using optical stimulation, and has a shape that is similar or identical to the shape of neural spike recording 1800.

In a demonstrative experiment, we then made neural recordings from multiple channels while blue laser pulses (at 473 nm) were directed through free space onto the OEM. Given the fabrication of the backside of the array (directly receiving the laser beam) and geometrical shadow masking due to patterning of the electrical contacts etc., the optically accessible regions were defined as approximately 150 μm optical apertures at each OEM element. As seen in the neural data of FIG. 16, clear and robust optically induced neural activity was evoked on several channels well above the spontaneous activity level. The culmination of these initial proof-of-concept experiments in acute rodent model represents critical initial demonstrations of the viability of the new wide-band gap semiconductor OEM concept.

Optoelectronic implants may be tested by extensive experimentation and assessment in freely moving rats. For freely moving rodents, the device can provide a new experimental neuroengineering toolkit in context of optical modulation of neural circuits, where the connection between such perturbations (e.g. as proxy for sensation of a forelimb of a rat, or elsewhere, a single digit of a non-human primate) can connect neural circuit dynamics to elucidate behavioral cause-and-effect relationships between sensory and motor action. In some embodiments, the optoelectronic implants may be suitable for implantation in, and use by, human beings.

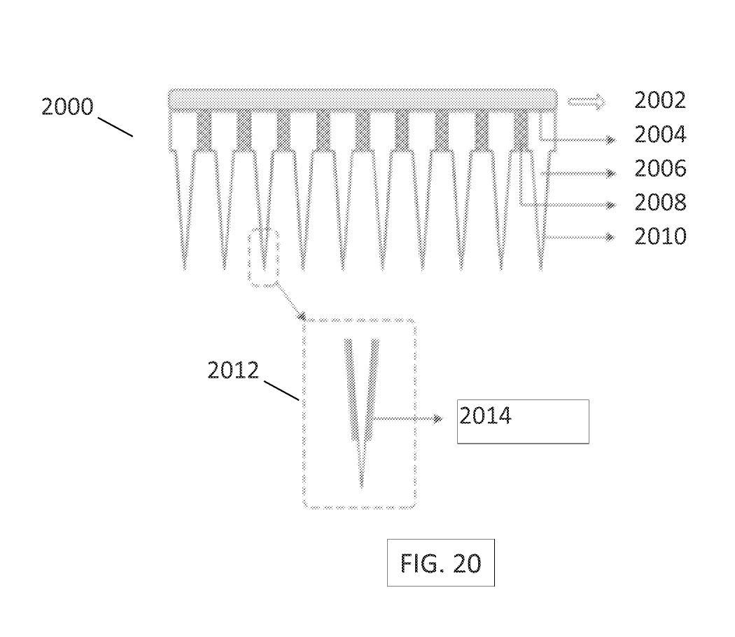

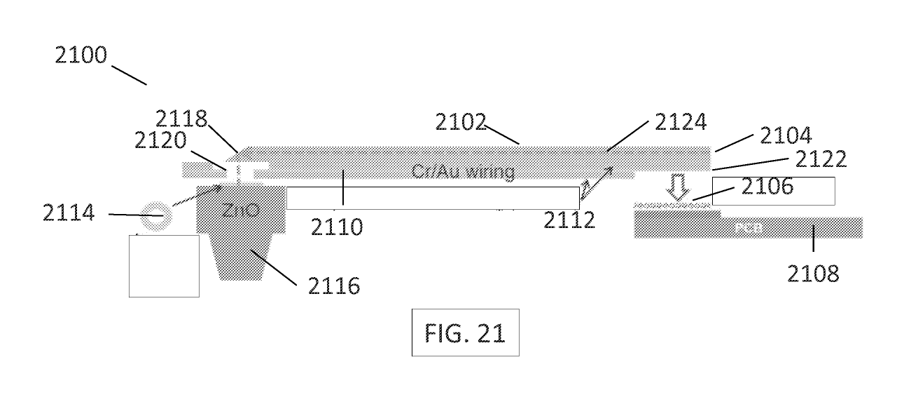

FIGS. 20 and 21 show engineering sketches of an optoelectronic microarray and a multichannel optical/electrical cable, respectively. While from device science and program development point of view the two components are easier to describe separately, they must of course be both integrable and to be physically integrated in final assembly.

### Chronic 100-Channel Wide-Band Gap Semiconductor Intracortical Optoelectronic Microarrays

FIG. 20 is a schematic diagram of an optoelectronic microarray, in accordance with some embodiments. OEM implant 2000 is shown with electrical readout 2002, metal contact 2004, ZnO optoelectronic microarray 2006, adhesive 2008, and parylene sheath 2010, in accordance with some embodiments. Inset 2012 is an inset of an individual electrode. Shaded area 2014 is an insulating coating, e.g., parylene sheath, in some embodiments.

In this section we focus on device development, assessment, and chronic use of the ZnO OEMs themselves. Initial results demonstrate optical neural cell activity modulation as discussed above and shown in FIG. 16 in an exemplary prototype array. A chronic intracortical 10×10 array with multichannel optical and electrical access is contemplated. In particular, the OEMs should demonstrate chronic stability and in vivo performance, e.g., long term (>1 year) biocompatible reliability. In development of a stable OEM, we can maintain the approximate form factor of the OEM as the (electrical-recording-only) silicon-based “Utah” MEAs, given the many demonstrations and recent breakthroughs with brain-machine interfaces with this intracortical array geometry in non-human primates and human neuroprosthesis. While undersampling the neural population, these types of single neuron-resolution spike-resolving tools can be coupled with powerful statistical algorithms to decode neural population and (state-space modeled) dynamics. Moreover, the geometry including the typical pitch of the inter-electrode separation lends itself well to optogenetic definition of a target zone largely within a single electrode element of an OEM.

Control of profile and surface quality of optoelectronic microarray elements. A number of chemical and physical etching methods were examined as candidates to process bulk ZnO into the desired OEM form factor, with individual tapered optoelectronic microarray elements up to 1-2 mm in length. While the initial results using a tailored choice of H₂SO₄ and/or FeCl₃ at specific concentrations and temperatures in a wet etching step were quite reasonable, it may be desired in some embodiments to achieve finessing of the etching processes in order to reach a desired control of anisotropy (i.e. taper angle) and surface smoothness for the needles forming the electrodes of the optoelectronic microarray.

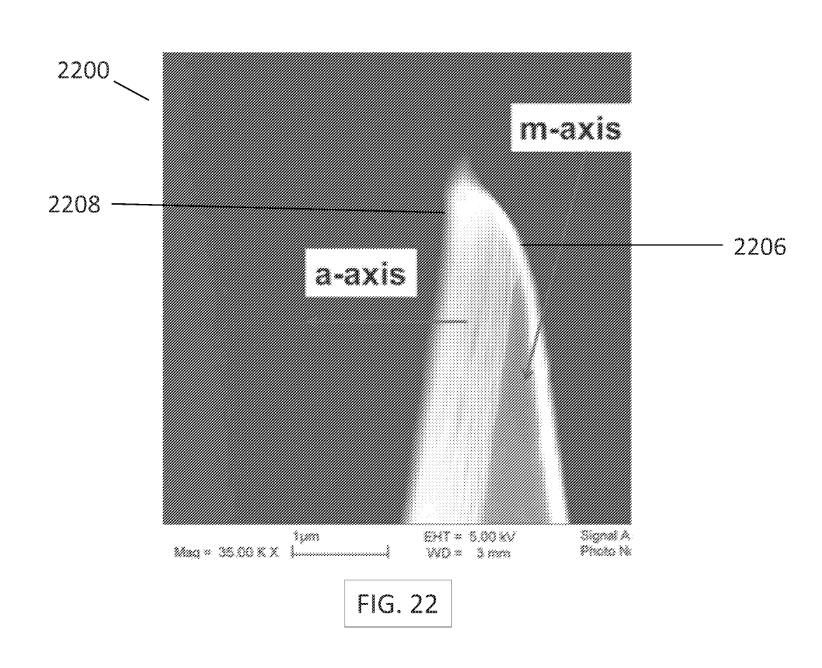

FIG. 22 is a micrograph of a wet-edged optoelectronic microarray tip, in accordance with some embodiments. Electron microscope image 2200 shows a close-up of a wet-etched ZnO optoelectronic microarray tip showing asymmetry in taper along the m- and a-axes of hexagonal ZnO. Two degrees of taper 2208, 2206 are visible in the figure.

FIG. 22 shows a close up view of an electron microscope image at the tip of on one ‘needle’ displaying the presence of the anisotropic etching on the micrometer scale. Given the c-axis growth direction of the bulk ZnO, we have found that the etch rates of the a- and m-facets are distinct. This tends to create a somewhat “blade-like” end to the tips as seen in the figure, with different degree of microscopic roughness along the two directions. While such asymmetry in itself is not necessarily detrimental (cf. “Michigan” MEAs), it is desirable to control this at micrometer-level precision. Suitable wet chemical etching conditions in terms of concentrations and temperatures can be selected to provide the optimal set of reproducible etch conditions as measured e.g. via broadband (1 Hz-1 KHz) electrode impedance spectroscopy. Note that finite micro-roughness has an effect on the electrode impedance (through increased surface area) at the semiconductor/electrolyte interface, with electrolyte referring mainly to the cerebrospinal fluid in the brain. Dry etching step by inductively couple plasma tool (ICP) for post-wet chemical smoothing can also be employed.

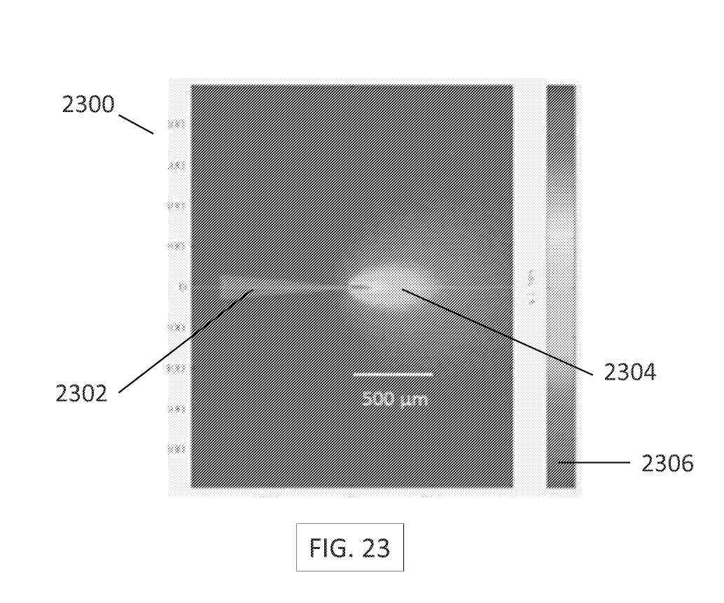

FIG. 23 is a simulated tip emission pattern, in accordance with some embodiments. Tip emission pattern 2300 is the output of a Monte-Carlo light scattering simulation in brain tissue of emission patterns from the tip of optical fiber waveguide 2302. Emission pattern 2304 is shown and log scale blue light intensity color code scale 2306 is shown. Emission pattern 2304 is characteristic of waveguide 2303, which has a tapered fiber with 10 μm exit aperture.

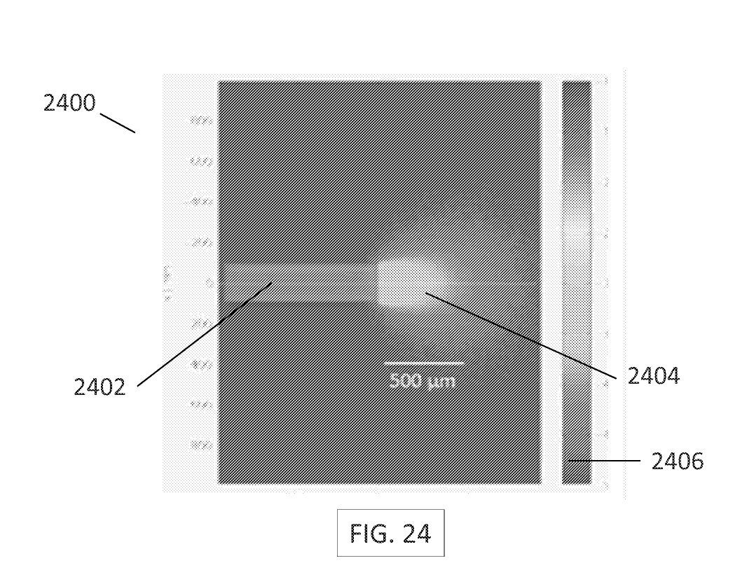

FIG. 24 is a further simulated tip emission pattern, in accordance with some embodiments. Tip emission pattern 2400 is the output of a Monte-Carlo light scattering simulation in brain tissue of emission patterns from the tip of optical fiber waveguide 2402. Emission pattern 2404 is shown and log scale blue light intensity color code scale 2406 is shown. Emission pattern 2404 is characteristic of waveguide 2403, which has a blunt fiber with a 200 μm aperture.

In order to complement and guide the design and the fabrication of the OEM elements for desired spatial delivery of illumination to the cortex, and to enable prediction of the spatial shape of the electrophysiological recording volume, numerical simulations for glass-based single coaxial optoelectronic microarray can be used. The optical light delivery patterns can be quite well modeled by Monte-Carlo approaches which take into account tissue scattering (dominant), opsin absorption, and background brain tissue absorption.

For circularly symmetric tapered glass (index of refraction n=1.52), FIGS. 23 and 24 show examples from such a computation for two different types of glass fiber optical apertures. For the ZnO OEMs, the ability in principle to control the degree of shape anisotropy of the light emitting tip from blade-like to a circular one, such simulations can help to tailor the optoelectronic microarray for “write-into” particular brain structures and their morphology by a given “beacon” of light formed at the tip. In terms or calculating the spatial directionality of the electrical extracellular recording of the circularly symmetrical, but conical coaxial o optoelectronic microarray, finite element electrostatic models have been developed in the PIs group, and will be deployed to take advantage of spatial anisotropies in designing the “read-out” directionality patterns of the new ZnO-based OEMs.

Chronic Material Stability and Biocompatibility. While noting that Zn2+-ion is not known to be toxic to body tissue, the OEMs can be assessed to determine their biocompatibility and chemical durability in brain tissue as follows. First, on bench top, the ZnO material in both planar and bulk form as well as in fully fabricated OEMs will be immersed to hot saline (T˜50-60 C) for accelerated testing of possible chemical corrosion effects over extended periods of time. After such immersion under controlled environment, the material will be analyzed by electron microscopy, and if needed, chemically, for any possible reaction products (say, with Cl-ions of saline). Second, like ZnO materials can be implanted into mice for chronic exposure for at least 6 months, following which morphological, chemical, and histological analysis can be performed.

Implant and Chronic Testing in Rats: The geometry and form factor of the proposed OEMs matches well with the surgical implant techniques which the PIs group has used over several years for different types of cortical implants, including both in vivo rodents and non-human primates (cf. list of relevant publications). Further, as part of the so-called single coaxial (Au-coated glass) optoelectronic microarray, we have also developed a protocol for monitoring the electrical and optical performance of the types of dual-function devices which the new OEMs will mirror. For example, monitoring the intensity of the fluorescent while using the OEM in an optical “imaging mode” can help to keep track of the location and health of the opsin expression level (even if indirectly). Likewise, the above mentioned impedance spectroscopy can be used as a tool to track the electrical recording performance of the OEMs, including inferences to possible microglia formation.

### 100-Channel Flexible Multichannel Electrical-Optical Dual Ribbon Cable

FIG. 21 is a schematic diagram of a planar ribbon cable, in accordance with some embodiments. Flexible optical/electrical planar ribbon cable 2100 is shown with SU-8 optical waveguide 2102, which includes optical write-in 2104 and milled edge 2118. Cr/Au wiring 2122 is shown sandwiched between two layers of polyimide dual cladding 2112; in some embodiments, cladding 2112 is roughly 40 μm in thickness. Cr/Au wiring makes contact with ZnO optoelectronic microarray tip 2116 at Au contact pad 2114, which has a ring shape. Gap 2120 permits optical signals to travel through Cr/Au wiring 2122 and cladding 2112, and the hole in the middle of Au contact pad 2114 permits optical signals 2124 (shown as dotted line) to travel from optical write-in 2104 through contact pad 2114 to optoelectronic microarray tip 2116. Reflection may occur at milled edge 2118; this may be due to internal reflection or may be due to additional mirroring effects of milled edge 2118. Cr/Au wiring 2122 is bonded using an ACF bond at bonding site 2106 to PCB 2108, such that electrical signals can be transmitted through PCB 2108 via wiring 2122 and contact pad 2114 to optoelectronic microarray tip 2116.

In order to transport and guide incident optical stimulation to specific elements of the OEM, and provide an electrical readout, likewise, from individual elements as a time-space resolved map of neural activity, relatively sophisticated and flexible cabling is needed. Such a cable can be, for example, connected to the intracortical OEM at one end, threads through the skull of the subject (as with comparable MEAs) and attaches at distal end either to a skull-mounted pedestal or future subcutaneous wireless implant which house the neural signal first stage read-out electronics and, now additionally, access to the blue-green pulsed light sources. This cable can be thought of as an umbilical for the intracortical OEM.

FIGS. 20-21 above showed the concept schematically for design of the light-electrical cable, designed to be scalable up to 100-channels, while retaining flexibility. The design is based on thin polyimide film base layer (˜40 μm) which embeds the high density of Au-planar wires for electrical read-out. Atop the electrical connector, with a very thin PDMS spacer layer, resides a transparent polymer (such as SU-8) layer which in turn defines the multichannel optical waveguide assembly. The PI's laboratory has systematically sought for and is familiar with the electronic and optical materials, including their microfabrication, which will be used as follows:

Multichannel polyimide electrical ribbon cable. Thin layers of polyimide are seeing widespread use as robust environments that require embedding of high density electrical wiring in number of hermetic biomedical implant applications. Polyimide, subject to process compatibility with other materials due to its somewhat high curing temperature, will be used in this project for the electrical component of the multichannel integrated input-output connector cable.

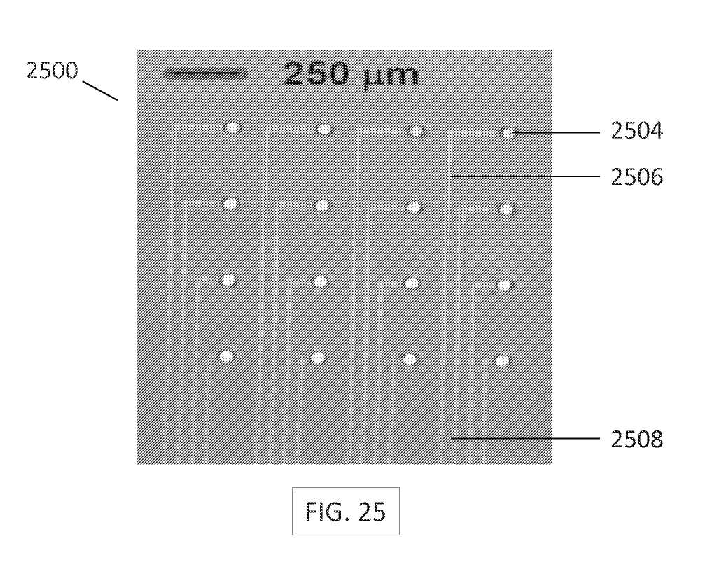

FIG. 25 is a top view of a section of a 16-channel polyimide test cable (20/300 nm Cr/Au) with fan-out to contact pads, in accordance with some embodiments. A multichannel integrated input-output connector cable is shown. Polyimide as mentioned in the present disclosure may include DUPONT™ KAPTON™ polyimide film, Apical, UPILEX, VTEC PI, Norton TH, Kaptrex, or another polymer of imide monomers. Top view 2500 shows fan-out 2508, which includes trace wire 2506, which is coupled to contact pad 2504. Contact pad 2504 can be electrically coupled to a single electrodes of the optoelectronic microarray, in some embodiments.

We have made 5-10 cm long test structures in preliminary work, with very low resistance, such as shown in FIG. 11 where approximate 25 μm wide Au-leads set in a 25 μm pitch have been sandwiched within a 40 μm thick highly flexible, yet mechanically robust cable (PI 2611). The figure shows a portion of such a cable where the fan-out from the cable to a connector (either OEM or distal end) is facilitated. In the proposed work, these cables will be scaled up to 100-channels and optimized for integration with the optical waveguide layers (see below). For the test structures such as in FIG. 11, we have ascertained the lack of cross-channel interference up to frequencies of several KHz.

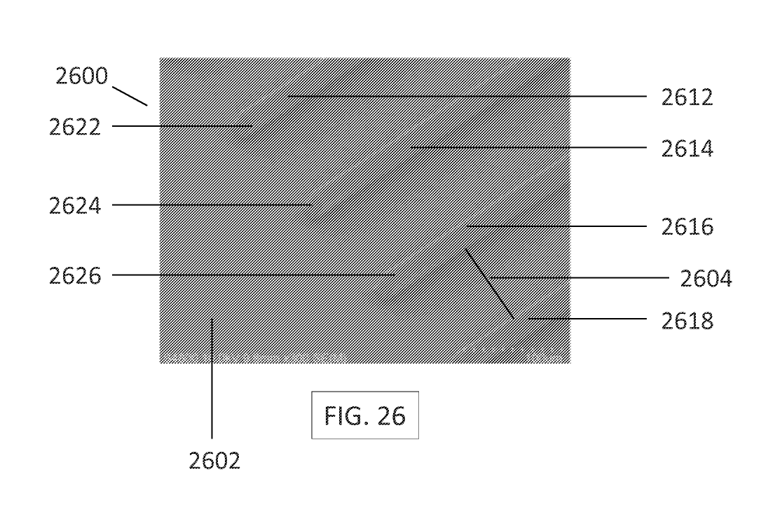

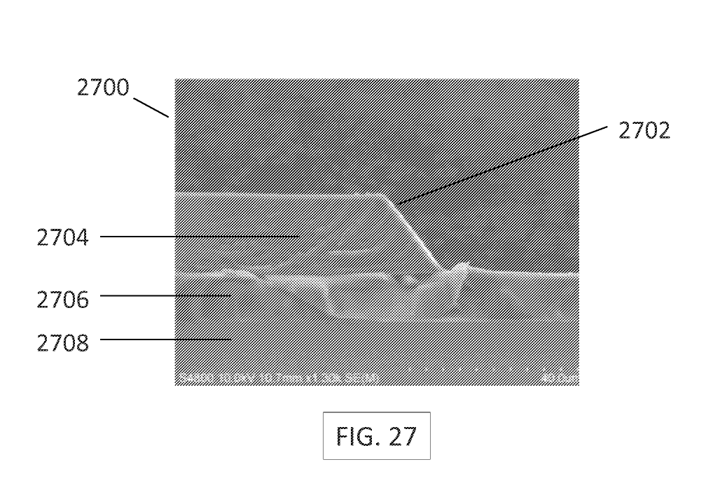

FIGS. 26-27 shows a sample of four parallel 20×20 micron SU-8 ridge optical waveguides in accordance with some embodiments, showing the angled end (here ˜50 degrees) for prismatic reflection of in-plane light into vertical downward direction of the ZnO OEM optical input aperture (left). A cross-sectional electron microscope view of the ion milled angle reflector is shown.

FIG. 26 is an isometric view of an optical waveguide, in accordance with some embodiments. In some embodiments, substrate 2602 is shown with four parallel 20×20 micron SU-8 ridge optical waveguides 2612, 2614, 2616, 2618, each terminating at an angled end 2622, 2624, 2626, (not shown). The distance between optical waveguides can be regular, shown here as 2604. In some embodiments, the angled end can be ion milled to 50 degrees for prismatic reflection of in-plane light into vertical downward direction of the ZnO OEM optical input aperture.

FIG. 27 is a cross-sectional micrograph of an optical waveguide, in accordance with some embodiments. Micrograph 2700 shows waveguide 2604 on top of substrate 2706 and 2708. Milled edge 2702 provides an angled end of waveguide 2604 for reflecting optical signals downward toward a ZnO OEM.

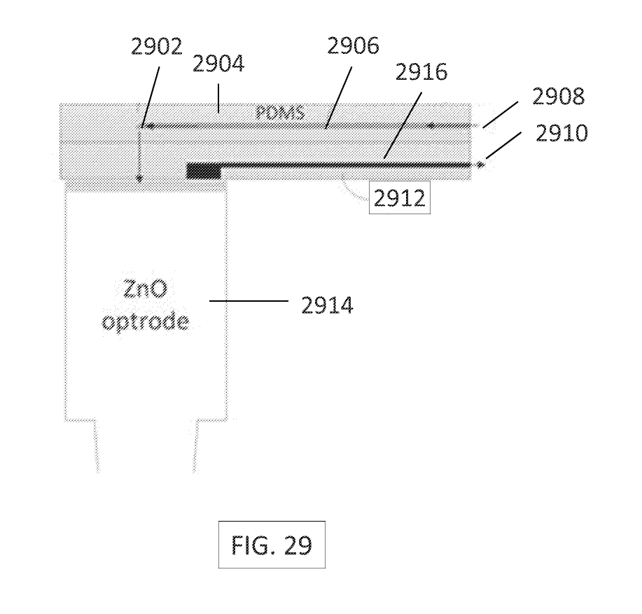

Multichannel Optical Waveguide Interconnect Cable. While the choice of a polyimide cable can be viewed as a very useful choice for electrical cabling, the flexible cable construct which also desirably accommodates a commensurate number of parallel optical ridge waveguides, while maintaining still compatibility with the planar ribbon geometry. In one approach transparent polymer waveguides are used, based on their optical transparency and relative readiness for microelectronic process approaches (such as SU-8). We have processed ridge waveguide arrays such as in FIG. 26, 27, where each waveguide is patterned to a suitable angle at the OEM end, so as to deflect the in-plane propagating blue-green light into the predetermined entrance aperture of a given element of the ZnO OEM array (see scheme of FIG. 29). We have estimated coupling losses in such a case to be acceptable (˜10%) when the array fabrication is optimized in the laboratory with precision lithographic alignment techniques.

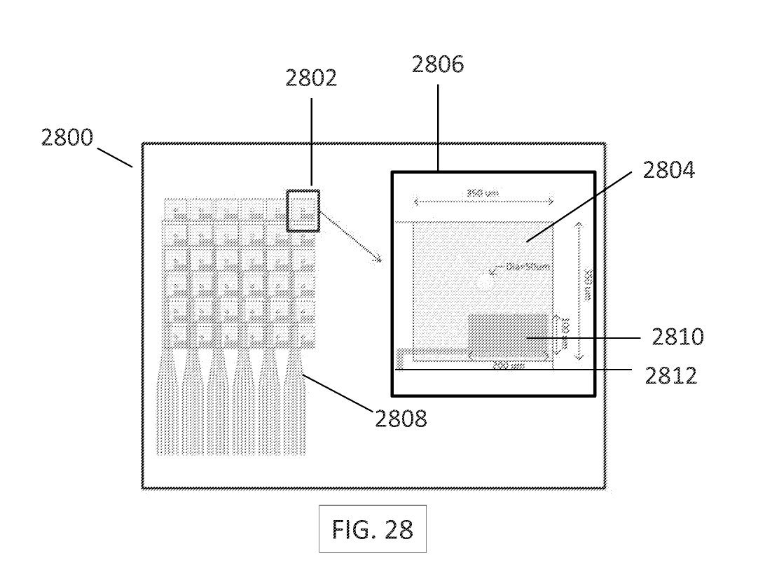

FIG. 28 is a schematic top view of an optoelectronic microarray electrical connection scheme, in accordance with some embodiments. Diagram 2800 shows a 36-channel electrode array with individual electrode 2802 and fan-out 3808. Inset 2806 shows gap 2804, electrical contact 2810, and wire 2812. Wire 2812 is coupled to fan-out 2808 and communicates electrical signals to a ZnO optoelectronic microarray (not shown) via electrical contact 2810, which is in communication with the optoelectronic microarray Gap 2804 provides an opening through which an optical waveguide (not shown) can send optical information through electrode 2806 to the underlying ZnO optoelectronic microarray.

FIG. 29 is a schematic cross-sectional view of an optoelectronic microarray electrical connection scheme, in accordance with some embodiments. ZnO optoelectronic microarray 2914 is connected to the optical and electrical assembly above it, and projects into surrounding brain tissue to directly stimulate neurons in the brain tissue and measure electrical impulses in neurons in the brain tissue. Wire 2916 is connected to electrical output 2910 to optoelectronic microarray 2914 through substrate 2912, such that electrical signals can pass through the optoelectronic microarray 2914 and wire 2916 to reach a processing card (not shown) via electrical output 2910. In some embodiments, electrical stimulation may be bi-directional. Optical waveguide 2906 may be any transparent waveguide (such as made of SU-8), and may pass through PDMS substrate 2904. Deflector 2902 allows optical signals to pass through optical waveguide 2906, be deflected in a downward direction, and pass through the ZnO optoelectronic microarray 2914 to stimulate brain tissue. Assembly and alignment of the integrated dual electrical-light cable. Special attention can be paid to the alignment and connection of the dual-function ribbon cable to the OEM arrays. As already suggested above, the light-in/electrical-out stacked planar cable will be been designed with a self-aligned feature firmly in mind, to facilitate the simultaneous connection between 100 optical and electrical elements, respectively. FIG. 13 shows schematically the present plan which will be pursued early in the project (36-channel version). Briefly, the Au-metallization defines a footprint at each element of the OEM which leaves an optical aperture of approximately 50 μm for entering the blue-green laser or LED light into the ZnO tapered optoelectronic microarray guide. We believe that this method of approaching this need will initially deliver at least “100-points of light”, and is scalable to thousands of points of light, into targeted brain circuits and reading out the associated circuit dynamics to complete the bidirectional cortical network interface.

### Integration of Light Sources With the Optoelectronic Microarray

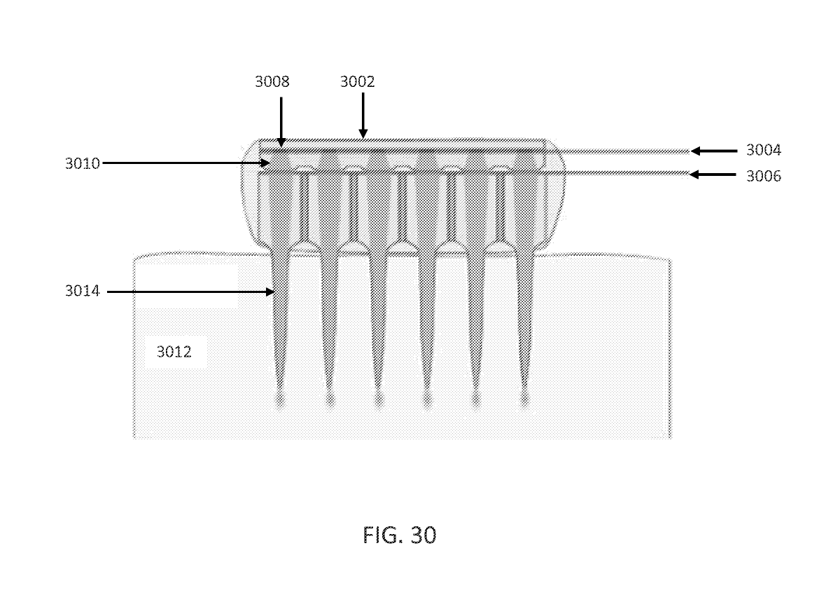

FIG. 30 shows an alternative embodiment that integrates compact light sources into the optoelectronic microarray. In this embodiment, rather than guiding light originating from an optical write-in through a flexible optical waveguide, light can be locally generated by light sources integrated into the optoelectronic microarray. Optoelectronic microarray 3002 can be connected to an incoming electrical wire 3004 and an outgoing electrical wire 3006. Optoelectronic microarray 3002 can comprise a plurality of micrometer-sized light-emitting diodes or laser diodes (hereinafter referred to as LEDs) 3008 capable of emitting colored light (e.g., blue, green, red, or white), as well as a plurality of microarray tips 3014 embedded in brain tissue 3012. Microarray tips 3014 are both optically transparent and electrically conductive, and can comprise wide band gap semiconductor materials such as zinc oxide, gallium nitride, and/or silicon carbide, as discussed above. The plurality of LEDs 3008 can be attached to or embedded into optoelectronic microarray 3002 in a planar 2-dimensional array structure, wherein each LED 3008 is positioned at the base of a corresponding microarray tip 3014. Such micro-LED array structures are known from, for example, Xu et al., J. Phys. D 41, 094013 (2008). LEDs 3008 can be connected to microarray tips 3014 through a microlens array 3010. Microlens array 3010 can comprise light condensers or other passive optical components configured to efficiently direct light from LEDs 3008 to the microarray tips 3014.

Incoming wire 3004 can supply electrical energy to individual LEDs 3008 to generate arbitrary spatio-temporally patterned light. The emitted light is guided to the microarray tips 3014 by microlens array 3010. Microarray tips then emit the light at the narrow aperture at its tip into brain tissue 3012. Recorded multichannel neural signals sensed by microarray tips 3014 are transferred to outgoing wire 3006, which can be separate from or bundled together with incoming wire 3004. Microelectronic packaging techniques such as flip-chip bonding between light source, optical component and optoelectronic microarray can be used to maintain separation between the optical and electrical pathways.

### Embodiment of the Bidirectional Neural Device as a Wireless System

In yet another embodiment, the entire device system can be made wireless by housing the electronics and optics in a single headmounted or implanted module. For example, instead of using incoming wire 3004 to transmit electrical energy to LEDs 3008, and outgoing wire 3006 to transfer recorded neural signals, a processor or other logic circuit as well as a radio frequency transceiver can be integrated directly with optoelectronic microarray 3002. In this embodiment, the radio transceiver can both transmit the recorded neural information to outside receivers, but also receive command signals for electrical activation of the LEDs 3008. This wireless embodiment can be useful for moving subjects and mobile applications where the use of physical wires can be impractical or disadvantageous.

The foregoing has outlined some of the more pertinent features of the subject matter. These features should be construed to be merely illustrative. Many other beneficial results can be attained by applying the disclosed subject matter in a different manner or by modifying the subject matter as will be described. For example, the size of the optoelectronic microarray may be decreased, or increased.

1. An optoelectronic device, comprising:
a plurality of electrodes secured to a common base to form an array, each electrode providing both optical transparency and electrical conductivity and each electrode electrically isolated from the others;
wherein each electrode is configured and arranged to act as a waveguide to transmit light from a first base proximal to the common base to a second tip distal to the common base.

2. The optoelectronic device of claim 1, wherein each electrode tapers from the first base proximal to the common base to the second tip distal to the common base.

3. The optoelectronic device of claim 1, wherein the electrodes comprise a wide band gap semiconductor material.

4. The optoelectronic device of claim 3, wherein the wide band gap semiconductor material comprises a material selected from the group consisting of zinc oxide, gallium nitride and silicon carbide.

5. The optoelectronic device of claim 1, wherein the second tip of the electrodes comprise a conductive coating.

6. The optoelectronic device of claim 1, wherein the electrode comprises an electrically insulating coating.

7. The optoelectronic device of claim 1, wherein the electrodes comprise an electrically insulating coating located to expose the tip of the electrode.

8. The optoelectronic device of claim 1, further comprising a plurality of electrical contacts disposed over the common base on a side opposite the array, each electrical contact in electrical connection with an electrode.

9. The optoelectronic device of claim 1, further comprising an electrical multichannel cable, each channel electrically connected to a unique electrical contact.

10. The optoelectronic device of claim 9, wherein an optical cable is co-located with the electrical multichannel cable.

11. The optoelectronic device of claim 10, wherein the optical cable comprises a plurality of waveguides, each waveguide optically connected to a unique electrode.

12. The optoelectronic device of claim 1, wherein the array comprises at least 25, electrodes.

13. The optoelectronic device of claim 1, further comprising a plurality of light sources secured to the common base to form a second array, each light source being positioned adjacent to the first base of a corresponding electrode.

14. The optoelectronic device of claim 13, wherein the plurality of light sources comprise a light emitting diode or a laser diode.

15. The optoelectronic device of claim 13, further comprising a plurality of lenses secured to the common base to form a third array, each lens being positioned to focus light originating from a corresponding light source.

16. The optoelectronic device of claim 13, further comprising a second electrical multichannel cable, each channel electrically connected to a unique light source.

17. A system capable of optical stimulation and electrical recording, comprising:
an optoelectronic device according to claim 1;
a flexible optical conduit providing individual optical connection from each of the electrodes in the array to an optical signal source;
a flexible electrical conduit providing individual electrical connection from each of the electrodes in the array for receiving an electrical signal;
a circuit assembly coupled to the electrical signal source and the optical signal source; and
a processor for providing control of at least one of the electrical signal source and the optical signal source.

18. The system of claim 17, wherein the flexible optical conduit comprises a plurality of waveguides, each waveguide configured and positioned to direct light from the optical signal source into a unique electrode.

19. The system of claim 17, wherein the flexible optical conduit and the flexible electrical conduit are co-located in a single cable.

20. A method of making an multielectrode array comprising:
forming a first set of channels in a first side of a wide band gap semiconductor single crystal to provide isolated islands;
filling the channels with an electrically insulating material to electrically isolate each island;
depositing an electrical contact on each electrically isolated island;
forming a second set of channels in a second side of the wide band gap semiconductor single crystal to provide isolated columns, said second set of channels disposed over and extending to a depth of the electrically insulating material; and
shaping the columns to form a taper from a base proximal to the electrically insulating material to a tip distal from the electrically insulating material.

21. The method of claim 20, further comprising coating the tapered columns with an electrically insulating material, wherein the tip is free of the insulating material.

22. The method of claim 20, further comprising coating the tip with a transparent electrically conducting material.

23. The method of claim 20, wherein the forming of the first and second set of channels is accomplished by dicing.

24. The method of claim 20, wherein the shaping of the second set of channels is accomplished by anisotropic etching.

25. The method of claim 24, wherein the anisotropic etching comprises wet etching or dry etching.
